(19) United States
(12) Patent Application Publication
(10) Pub. No.: US 2008/0125725 A1
(43) Pub. Date: May 29, 2008

(54) Syringes and IV infusion systems for clinical trials

(76) Inventor: Nabil Dib, Phoenix, AZ (US)

Correspondence Address: Squire, Sanders & Dempsey L.L.P.;Two Renaissance Squire, Suite 2700, 40 North Central Avenue, Phoenix, AZ, 85004-4498, US

(21) Appl. No.: 11/499,041

(22) Filed: Aug. 4, 2006

(51) Int. Cl. A61M 5/00; A61M 37/00; A61M 31/00; A61M 5/31

(52) U.S. Cl. 604/264; 604/85; 604/93.01; 604/240

(57) ABSTRACT

The invention is a medical device for containing and/or dispensing a therapeutic agent or a placebo to a patient. The medical device is preferably a syringe or an intravenous (IV) infusion system. A device according to the invention has a viewing portion through which a therapeutic agent or placebo contained therein may be seen by a patient or operator. The viewing portion is colored so that a therapeutic agent having a first color and a placebo having a second color that is different from the second color are virtually or entirely indistinguishable when contained within the device and viewed through the viewing portion.

### TECHNICAL FIELD

The present invention relates generally to the field of medical devices, and particularly to syringes or intravenous infusion systems (collectively, “devices”) for containing and/or administering a therapeutic agent or a placebo to a patient. The therapeutic agent or placebo is in a liquid form that can be injected into a patent. A device according to the invention has a viewing portion through which a therapeutic agent or placebo contained therein may be seen by a patient or operator. This portion is colored so that a therapeutic agent having a first color and a placebo having a second color that is different from the second color are virtually or entirely indistinguishable in appearance when contained in the device and viewed through the viewing portion.

### BACKGROUND OF THE INVENTION

In clinical trials of therapeutic agents, for example pharmaceuticals or cell cultures, patients are placed in either a control or test group. Those in the test group receive a therapeutic agent, while those in the control group receive a placebo. For clinical trials to be most successful a patient should not know if he/she is receiving the therapeutic agent or the placebo, because such knowledge can influence trial results. Further it is important that an operator (e.g., doctor, nurse, technician or clinician) who administers a treatment not know whether a patient is receiving therapeutic agent or placebo, because a patient can also gain knowledge from the behavior of the operator and/or the operator may unconsciously skew the data related to the trial due to his/her knowledge.

Many therapeutic agents (which may be cloudy or milky in color) are visually distinguishable from placebos, which are often colorless (or clear). This is particularly true for cell culture solutions used in cell therapy. In these cases a patient and/or operator would be able to tell a placebo from a therapeutic agent based on the visual appearance of the solution used.

Therapeutic agents and placebos are often dispensed to patients by injection using a syringe or delivered via an intravenous infusion system. Syringes used in medical settings are typically disposable and transparent. Similarly, the intravenous (IV) bag, IV line and other parts of an intravenous infusion system are generally made of transparent flexible, polymeric materials. Transparent syringes and IV infusion systems allow for the visual appearance of the contents to be readily observed through the walls, for example, of the syringe barrel, IV bag or IV tubing. When such devices are used to dispense, for example, a cell culture solution that is milky or cloudy the visual appearance of this therapeutic agent is readily discernable and distinguishable from clear placebo.

As used herein, “therapeutic agent” means any material that can be injected into a patient through a syringe or IV system and that is supposed to render a beneficial physiological effect. Examples of therapeutic agents include, but are not limited to, synthetic and natural inorganic and organic compounds, proteins, DNA and RNA nucleic acid sequences, cell cultures, and gene cultures.

“Placebo” means any material that can be administered to a patient through a syringe or IV system and that is not supposed to render the same effect as a therapeutic agent used in the same trial. A placebo is often a saline solution, dextrose solution, glucose solution, or a mixed solution of multiple electrolytes and dextrose such as PLASMA-LYTE 56™ and 5% Dextrose solution (available from Baxter International, Inc., Deerfield, Ill.).

“Color” means any color and includes milky, brownish, greenish, bluish, clear and any other color. A viewing portion according to the invention has one or more colors (but cannot be completely clear) but a person may still see therapeutic agent or placebo through the viewing portion.

### SUMMARY OF THE INVENTION

Medical devices of the present invention include syringes and one or more components of an intravenous infusion system. A medical device according to the invention is for containing and/or administering a therapeutic agent having a first color or a placebo having a second color that is different from the first color. The device has a viewing portion through which the therapeutic agent or placebo can be seen. The viewing portion has one or more colors, or has a surface coloring or pattern (collectively, “colored”) so that a person is able to see through the viewing portion, but unable to visually distinguish between, a therapeutic agent having a first color and a placebo having a second color that is different from the first color. For example, the viewing portion of a medical device of the present invention may be translucent and milky in color. In that case, a person could not visually distinguish between, for example, a cell culture that is milky in color and a placebo that is clear, when each is seen through the viewing portion.

### BRIEF DESCRIPTION OF THE DRAWINGS

FIG. 1 illustrates one embodiment of a syringe of the present invention.

FIG. 2 illustrates one embodiment of an intravenous infusion system of the present invention.

### DETAILED DESCRIPTION

Medical devices disclosed herein are useful for containing and/or administering a therapeutic agent or placebo in clinical trials wherein the therapeutic agent has a first color and the placebo has a second color that is different from the first color. The medical device may be a syringe or an intravenous (IV) infusion system and has a viewing portion through which the therapeutic agent or placebo may be seen. As used herein “a viewing portion” means one or more viewing portions. For example, in an intravenous infusion system, the viewing portion may include all or part of the IV bag and all and the IV line. The viewing portion is colored so that when placed in the device and viewed through the viewing portion, the therapeutic agent and placebo are virtually or entirely indistinguishable in appearance (or also referred to herein as “visually indistinguishable or the visual appearance is “the same”).

Optionally, any of the embodiments of the present invention may have a textured surface to assist in camouflaging the visual appearance of the therapeutic agent or placebo inside of the device. A device of the present invention may have multiple viewing portions of the same or multiple colors, textures, or degrees of translucency.

The medical devices of the present invention may be disposable or reusable, can be formed of one or more suitable materials, such as glass or polymeric materials, and the methods of making such devices are known. For example, IV bags and other components of an intravenous infusion system of the present invention are preferably formed of one or more flexible, biocompatible polymeric materials that are flexible. Examples of suitable materials include, but are not limited to, polyolefins such as polyethylene or polypropylene, polyvinyl chlorides, and ethylene-vinyl acetates copolymers. Selected components of the devices, such as needles, may be formed of a metal, metal alloy, ceramic, or other suitable material.

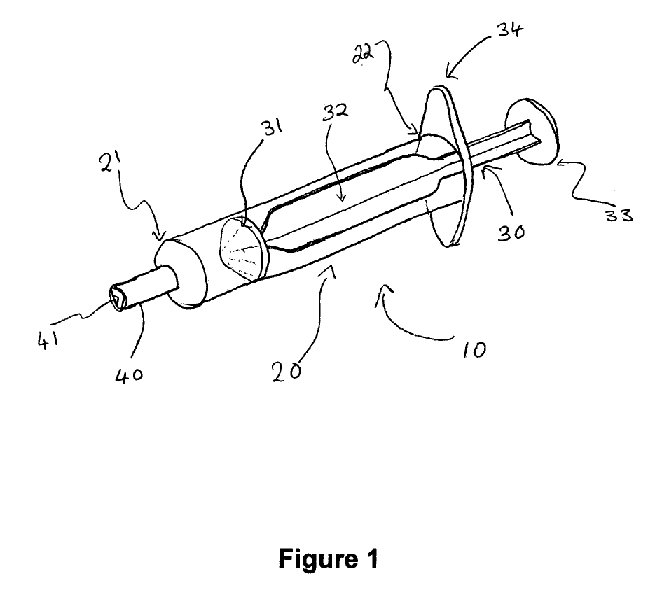

The medical device may be a syringe comprising a barrel through which the solution may be seen. A syringe of the present invention comprises a barrel, a plunger assembly, and means for attaching a needle or cannula. FIG. 1 illustrates one embodiment of a syringe useful in practice of the present invention. The syringe (10) comprises a barrel (20), a plunger assembly (30), and means for attaching a needle or cannula (40). The barrel (20) has two ends: a distal end (21) and a proximal end (22). The syringe barrel of the present invention is typically cylindrical, although any other tubular shape may be used. The shape and volume of the barrel can vary significantly depending on the application and dose to the administered. The syringe barrel can be of any bore, length, or volume suitable for the particular application.

In this embodiment, the proximal end (22) of the barrel is open and adapted to receive a plunger assembly (30). The plunger assembly (30) fits tightly inside the barrel and comprises a sealing means (31), a plunger rod (32) and a thumb-press (33). The length of the plunger assembly (30) is greater than the length of the barrel (20). The sealing means (31) engages the proximal end of the barrel creating a liquid tight reservoir inside the syringe. Any type of sealing means know in the art may be used with the syringes of the present invention. The thumb-press (33) at the end of the plunger assembly (30) protruding from the barrel is used in cooperation with the finger-press (34) on the barrel to move the plunger assembly (30) to expel liquid from or to draw liquid into the syringe barrel. Any type of suitable plunger assembly can be used in the syringes of the present invention.

The distal end (21) of the syringe barrel is narrowed and provided with a means for attaching a needle or cannula (40). The needle attaching means (40) comprises an opening (41) through which liquid is expelled from the syringe barrel. The cannula or needle can be attached to the attaching means by any suitable method. For example, types of attaching means include, but are not limited to, luer fit, luer tip, luer lock, slip tip, or cementing. The needle may be integral with an adapter that is pushed or screw threaded onto the needle attaching means (40) as is known in the art. A needle with an adapter may also be inserted into opening (41) in the needle attaching means (40). The needle may be permanently attached or may be removable from the syringe barrel. A syringe of the present invention can be used with any suitable type of needle. The size of the needle or cannula will depend on the volume of the syringe and its application. Needles or cannula useful in the practice of the present invention may be of any suitable length, diameter, or bore dimension.

The syringe barrel of the present invention may further comprise calibration or volume measuring markings along the length of the barrel body to indicate, for example, the volume of liquid within the syringe. Any form of calibration marking may be used, such as, for example, volume (ounce or cubic centimeter (cc)), and dose. The viewing portion for a syringe according to the invention would include all or part of the barrel.

While the syringes of the present invention have been described with reference to FIG. 1, other syringe designs may also be used.

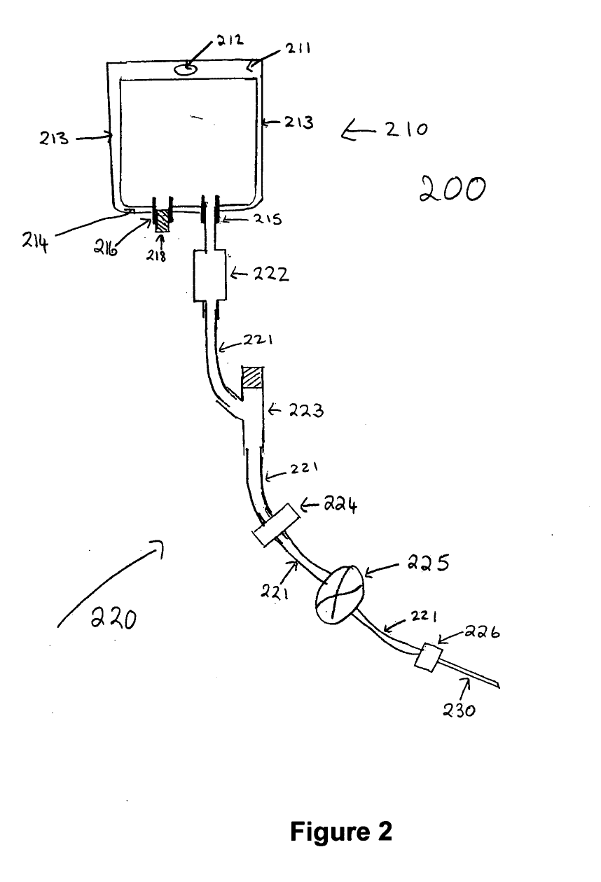

An intravenous infusion system of the present invention comprises an IV infusion bag and IV line with means to infuse a fluid into a patient. FIG. 2 illustrates one embodiment of an intravenous infusion system useful in practice of the present invention. The intravenous infusion system (200) comprises an IV infusion bag (210), an IV line (220) and a needle or cannula (230). The IV bag (210) contains the solution to be infused into the patient, and is usually made of suitable, biocompatible, flexible polymeric material, although semi-ridged or glass containers can also be used. IV bags are usually formed from two flexible plastic sheets, and any shape or construction suitable may be used. One end of the IV line (220) is attached to IV bag (210) in communication with the fluid in the bag, and the other end of the IV line is attached by connector (226) to a needle or cannula (230) that is used to infuse the fluid into a patient. The IV bag (210) comprises top seam (211) usually with a device (212) for hanging the bag or elevating it above a patient, two side seams (213) and a bottom seam (214). The bottom seam (214) of the bag has at least one exit port (215) with a connector for connecting an IV line (220) through which fluid can be extracted into an attached IV line. The bag may further comprise one or more other openings or ports in the bottom seam of the bag (216) with an injector (218) for injecting fluid into the bag. The bag may also comprise other openings in the side or top seems or on a bag wall. The ports for use in the intravenous infusion systems of the present invention can be a tube or other type of exit or injecting port. For example, in one embodiment, a port is a tube which is inserted and sealed between the back and front bag walls either by means of being formed integrally with the walls or by means such as welding, bonding, gluing or another permanent seal. Each port preferably has a seal. The seal can be of any suitable type such as a membrane, resealable membrane, or other device such as, for example, a value or a universal connector as described in U.S. Pat. No. 6,602,239 to Rönneklev. The shape and volume of the IV bag can vary depending on the application and dose to be administered.

The IV line (220) comprises one or more lengths of tubing (221), a means for attaching a needle or cannula (226), and optionally one or more fittings such as, for example, a drip chamber (222), an elbow (223), a connector (224), and a value or other flow regulation device (225). One end of the IV line is attached to the exit port (215) by any suitable structure, such as an IV spike. The drip tube via an IV spike may be directly connected to the exit port (215), as shown in FIG. 2. The other end of the IV line is attached by connector (226) to a needle or cannula (230). The needle or cannula may be of any type or size suitable, and may be formed of metal or polymeric material. The IV line may further comprise a device, such as a roller clamp or valve (225), for regulating fluid flow through the IV line.

While the intravenous infusion device of the present invention has been described with reference to FIG. 2, other infusion system designs may also be used. A viewing portion for an IV infusion device according to the invention could include all or part of the IV bag and/or all or part of the IV line and/or part of the needle or cannula.

While particular embodiments of the present invention have been described, changes and modifications can be made without departing from the spirit and scope of the teachings and embodiments of this invention. The specification and examples are thus exemplary only, with the true scope and spirit of the invention being set forth in the following claims and legal equivalents.

What is claimed is:

1. A medical device for containing a therapeutic agent having a first color or a placebo having a second color that is different from the first color, the medical device having a viewing portion through which therapeutic agent or placebo contained in the medical device may be seen, the viewing portion being colored so that the visual appearance of the therapeutic agent is the same as the appearance of the placebo when viewed through the viewing portion.

2. The medical device of claim 1, wherein the medical device comprises:
(a) a means for containing the therapeutic agent or placebo; and
(b) a means for administering the therapeutic agent or placebo to a patient.

3. The medical device of claim 1 that is a syringe.

4. The medical device of claim 3 wherein the syringe has a barrel, the viewing portion comprising at least part of the barrel.

5. The medical device of claim 1 that is an intravenous infusion system.

6. The medical device of claim 5 wherein the intravenous infusion system has a bag for containing therapeutic agent or placebo and an IV line, the viewing portion comprising at least part of the bag.

7. The medical device of claim 6 wherein the viewing portion comprises at least part of the IV line.

8. The medical device of claim 6 wherein the viewing portion comprises at least part of the bag and at least part of the IV line.

9. The medical device of claim 1 wherein the viewing portion is milky in color.

10. The medical device of claim 1 wherein the viewing portion is brownish in color.

11. The medical device of claim 1 wherein the viewing portion comprises multiple portions.

12. The medical device of claim 1 that is disposable.

13. The medical device of claim 1 that is reusable.

14. The medical device of claim 1 wherein the color of the viewing portion is selected based upon the color of one or more of the group consisting of: the therapeutic agent and the placebo.

15. The syringe of claim 3 that comprises a plunger assembly and a needle.

16. The syringe of claim 3 wherein the barrel is comprised of plastic or glass.

17. The intravenous infusion system of claim 6 wherein the bag and the IV line are comprised of plastic.

18. The medical device of claim 1 wherein the viewing portion is one color.

19. The medical device of claim 1 wherein the viewing portion is multiple colors.
